(19) United States
(12) Patent Application Publication
(10) Pub. No.: US 2005/0069514 A1
(43) Pub. Date: Mar. 31, 2005

(54) Gelatin encapsulated toiletry products

(76) Inventor: Anthony T Maleedy, Somerset (GB)

Correspondence Address: DINSMORE & SHOHL LLP, ONE DAYTON CENTRE, ONE SOUTH MAIN STREET, SUITE 1300, DAYTON, OH, 45402-2023, US

(21) Appl. No.: 10/503,649

(22) PCT Filed: Feb. 10, 2003
(86) PCT No.: PCT/GB03/00597

(30) Foreign Application Priority Data
Feb. 8, 2002 (GB) 0202980.9

(51) Int. Cl. A61K 7/075; A61K 7/08

(52) U.S. Cl. 424/70.24

(57) ABSTRACT

A tablet from of bubble/foam bath product comprising: A surfactant; gelatin; and a non-formaldehyde preservative, wherein the product is substantially free from formaldehyde.

### BACKGROUND OF THE INVENTION

1. Field of the Invention

The present invention relates to toiletry products and more particularly to toiletry products that are manufactured in shaped forms as distinct from being in the form of a powder or liquid, for example. The invention is more particularly concerned with providing a bubble or foam bath product in the form of a shaped tablet as distinct from the commonly known bubble/foam bath product which is generally in liquid form.

There is disclosed in United Kingdom Patent Application No 2 280 906 A in Example 5 a shaped bath gel product. The formulation in that Example 5 has proved in practice to be deficient in meeting the performance requirements of a tablet form bubble/foam bath product As indicated earlier bubble/foam bath products usually take the form of a liquid or possibly a powder or crystals. This is so that they will dissolve relatively rapidly in bath or shower temperature water and provide the desired bubble or foam phenomenon for the user.

In contrast soaps, although also available in disposable cream or liquid form, more commonly take the form of a tablet which may be opaque or translucent. The performance requirements of soap are essentially different from those of the bubble/foam bath product discussed earlier. With soap what is required is relatively slow dissolution in water so that the soap tablet can be used many times and will last for a relatively long period.

As well as the performance requirements associated with the actual use of the product there are also performance requirements associated with the shelf life of the pmduct, i.e. its preservation over time. Furthermore, there can also be performance requirements associated with the marketing of the product.

These various different performance requirements can and do give rise to conflict in terms of arriving at a product formulation which is satisfactory.

Cosmetic products require an adequate amount of preservative to prevent attack by moulds and bacteria. Bacteria or fungal attack can produce opacity in products that are clear, separation in emulsions and pearlescent products and can cause changes in both perfume and colour systems. Fermentation can also occur causing a complete breakdown of the product rendering it not only totally useless, but in some cases dangerous.

Preservation is a matter of selecting a suitable preservative for the individual product and in accordance with the legislative requirements in the country of sale. Preservatives should be chosen with the utmost care for the following reasons:

Their properties can be inhibited by the various surfactants in the composition.

They may not be stable and so may lose their effectiveness.

They may discolour the product

They may cause irritation of the skin or mucous membranes.

In order to meet the earlier discussed sometimes conflicting performance requirements the inventor has carried out extensive research

In order to optimise a formulation for a tablet form of bubble/foam bath product. One focus for this research was in the area of determining a preservative which would satisfy the “shelf-life” performance requirements whilst not adversely affecting the operational performance of the tablet.

Another focus of the research was also concerned with the “shelf-life” of the product in terms of any tendency to dry out or dehydrate and thus render the product unmarketable.

According to the present invention a tablet form of bubble/foam bath product comprising:

a surfactant;

gelatin; and

a non-formaldehyde preservative,

characterised in that the product is substantially free from formaldehyde.

Optionally but preferably the product also includes one or more of:

water;

foam booster/stabiliser;

humectant/moisturiser;

an anti-ingester;

a colouring;

a fragrance.

After testing and rejecting very many preservative systems, parabens have been identified as the preservatives of choice. Parabens are esters or p-hydroxybenzoic acid and are highly effective antimicrobials. They are safe, non-irritant, non-toxic and non-sensitizing, stable, non-volatile and biodegradable.

There is a series of parabens and each one in this series has a marked bacteriostatic and fungistatic activity at low concentrations against a variety of micro-organisms. In the product of the present invention preferably a combination of five parabens is used to obtain the optimum performance. These are methylparaben, ethylparaben, butylparaben, propylparaben, and isobutlparaben. These parabens are combined with phenoxyethanol to form the product ‘Phenonip’. Phenonip is supplied by Nipa Laboratories. Other similar paraben/phenoxyethanol solutions may be used in place of Phenonip, such as Phenochem supplied by Sharon Laboratories of Israel.

A major consideration in choosing the parabens for the product of the present invention, apart from their efficacy as preservatives, is the fact that they do not react with any other ingredient within the product. The possibility of adverse reactions has been a major drawback in using other preservatives, particularly formaldehyde/formalin, as they react with gelatin rendering the product ineffective.

Formaldehyde in the form of a 38 to 40% solution known as formalin, is widely used as a preservative. Despite the introduction of many other preservative systems. formaldehyde is still one of the most effective because of its broad-spectrum activity at low concentrations. Other major factors in its popularity are its low cost and its solubility in water, making it simple to incorporate in water based systems. Because of these benefits formaldehyde tends to be the preservative that a skilled person in the toiletries field would use. However, the present inventor's research revealed that formaldehyde was not a suitable preservative for the product of the present invention.

It was found that formaldehyde is capable of reacting with side-chain groups present on the gelatin molecule. This causes a cross-linking and branched chained molecules and results in increased viscosity and eventually the formation of a three-dimensional insoluble network.

Cross-linking increases the gel melting point, retards gelatin solubility in hot water, reduces swelling and increases the hardness of the gel. When formaldehyde is incorporated into the gelatin solution the viscosity increases and, just prior to the formation of the gel network, the solution becomes viscoelastic. This is evidenced by the solution climbing up the shaft of a stirrer. The gel will have a high melting point and may become thermally irreversible.

If formaldehyde is used as the deliberately added preservative in the product of the present invention, or even as the preservative already contained in any of the individual ingredients which go to make up the product of the present invention, the effect will be to prevent the product from dissolving in bath water at a temperature which is comfortable and acceptable. If the gel does not dissolve, no surfactant is released into the water and foam, therefore, is not produced. The product will remain at the bottom of the bath water with its shape intact.

A concentration of as little as 0.04% formaldehyde is believed to adversely affect the solubility of the product. For example, this concentration could arise when the main surfactant (comprising 50% w/w) of the product is sodium laureth sulphate, and this contains 0.2% formalin (40% solution of formaldehyde).

Gelatin is a crucial ingredient in the product of the present invention. It is gelatin which allows the product to take on a gel form which is uniquely thermo-reversible. The gels formed with gelatin are elastic in character, unlike the brittle gels obtained with other gelling agents such as agar, alginates and carrageenan.

Gelatin is a protein and, in common with all proteins, is made up of amino acids joined together by peptide linkages to form polymer chains. It is these polymer chains which give gelatin gels their unique characteristics, but which also make these gels vulnerable to reactions from polyfunctional reagents possessing reactive groups such as aldehydes, particularly formaldehyde.

Gelatin is non-toxic and non-irritant to normal skin and eyes, and forms stable, elastic gels which are thermoreversible.

Lime processed gelatins are slightly more stable than acid processed gelatins, particularly in relation to pH values. The gel bath shapes have a pH of approximately seven (making the product neutral). If, for any reason, the pH is lower (below five) then the rigidity of the gel decreases. This decrease is significantly sharper with an acid processed gelatin than with a lime processed one.

Although any gelatin may be used in the product according to the invention, it is advantageous to use a gelatin that has been manufactured by alkali-treatment of collagen, as gelatins produced by alkali-treatments are in general more pure than gelatins produced by acid treatment of collagen and therefore give rise to stronger more stable gels. It is preferred to use a gelatin having a Bloom strength of not less than 200 g. Gelatins having a Bloom strength in the range of 230 to 270 g, preferably, 250 g, give especially advantageous results.

The term Bloom strength is used herein in relation to gelatin to indicate the gel strength, that is the force (expressed in grammes) required to depress the surface of six 213% w/w gel, matured at ten degrees centigrade for sixteen to eighteen hours, a distance of 4 mm using a flat-bottomed plunger 12.7 mm in diameter.

When the gelatin is mixed with hot water it dissolves and, on cooling, the mixture sets as a gel, consisting of continuous aqueous and gelatin phases. It is believed that, on setting, a rearrangement of the individual gelatin molecules occurs, giving chain segments that are helical in configuration providing a strong but elastic structure. Other substances, for example, surfactant molecules, that may be present in the gelatin solution before setting become trapped in the gel structure.

The gelatin may be present in an amount of up to fifteen percent, for example, from four to fifteen percent, by weight, based on the total weight of gelatin, water and surfactant. For the product of the present invention, it is preferred for the gelatin content to be so chosen that the product dissolves rapidly when it comes into contact with warm water in use. In that case, the gelatin content is advantageously not greater than ten percent, and preferably not greater than five percent by weight, based on the total weight of gelatin, water and surfactant, with a gelatin content of four to four point five percent (4.5%) being especially preferred. This lower range of gelatin content has been found to provide enhanced solubility of the product in bath water without making the product unstable during storage.

The amount of water will generally be from ten to eighty percent by weight, based on the total weight of gelatin, water and surfactant. The actual amount of water to be used should be chosen having regard to the need to form a stable gel having the desired dissolution characteristics in water.

For bath gels and shower gels, it is preferred that the product contains from one to four parts water per part gelatin although in one embodiment of the invention (Example 2 described later) there is no specifically introduced water content.

It will be appreciated that the gel must be sufficiently strong for the product to retain its shape at room temperature. On the other hand, the stability of the gel should be such that the product of the invention has the desired dissolution characteristics. It is preferred for the gel to be such that the product dissolves rapidly, for example, during one normal usage, for example during one bath. The relative amounts of the principal components of the gel and in particular of the gelatin, water and surfactant are in that case preferably so selected that the product will dissolve in not more that five minutes, and preferably not more that three minutes, when in warm water, for example of temperature thirty-five to forty-five degrees centigrade in not more that six minutes and, advantageously, not more than three minutes.

The surfactant or surfactants present in the shaped product of the invention may or may each be an anionic, amphoteric, nonionic or cationic surfactant.

Examples of suitable anionic surfactants are alkali metal salts of organic compounds having alkyl radical containing from eight to twenty-two carbon atoms and a sulphonic acid or sulphuric acid aster radical. The alkyl radical may, if desired, be substituted by one or more substituents and/or interrupted by one or more hetero atoms, provided that any said substituents and hetero atoms present do not interfere adversely with the surfactant activity of the surfactant.

Sulphosuccinate, which is a mild anionic surfactant, may be used as the, or as a proportion of the, surfactant. When sulphosuccinate is used in combination with other surfactants, a synergistic effect is generally observed, the product being milder than might otherwise have been expected from consideration of the surfactant content of the product.

The product of the invention may, in addition to or instead of an anionic surfactant, contain one or more further surfactants selected from cationic, amphoteric, zwitterionic and nonionic surfactants.

Amphoteric surfactants that may be used in the shaped products of the invention include derivatives of aliphatic secondary and tertiary amines in which the aliphatic moieties may be straight chain or branched and in which there is an aliphatic moiety having from eight to eighteen carbon atoms and an aliphatic moiety containing an anionic water solubilizing group, for example, a carboxy, sulphonate or sulphate group. Examples of amphoteric surfactants that may be used in accordance with the invention are betaines, for example, cocoamidopropyl betaine.

Amphoteric surfactants generally have a lower solubilising activity than, for example, anionic surfactants, and therefore have less good cleansing properties. Certain surfactants having a very good cleaning properties, however, are found in use to result in undesirable drying of the skin and, where such surfactants are used, it will be advantageous to use instead of that surfactant alone a proportion of that surfactant in combination with an amphoteric surfactant, which by virtue of the milder action of the amphoteric surfactant reduces the extent of such drying. For example, betalnes may advantageously be used in hair shampoos in combination with an anionic surfactant or in hair conditioners in combination with a cationic surfactant. In addition to having surfactant properties and good conditioning properties, betaines have viscosity-increasing properties which, in the product of the invention, assist in retaining the shape of the product during storage.

The amount of surfactant is preferred to be at least fifty percent by weight, more preferably at least sixty percent by weight, based on the total weight of the product.

Sodium laureth sulphate is the most widely used anionic surfactant (surface active agent) in shampoos, shower gels and bubble bath products. It is used as a cleaning and foaming agent. It is clear, colourless and easy to work with. The preferred concentration used in the present invention is twenty-eight percent, the balance being water. This high level of water is necessary to allow the gelatin enough liquid to absorb and dissolve. In the present invention it is mainly this substance which, when released as the gel melts, creates the lather. The sodium laureth sulphate may itself contain a preservative, but in accordance with the present invention, this preservative does not include formaldehyde.

In addition to a surfactant and a gelling agent, the product of the invention preferably contains at least one humectant. The presence of a humectant is desirable to hinder the loss of moisture from the gel. The product preferably contains at least one humectant. The humectant is preferably selected from polyhydric alcohols, for example, glycerine and propylene glycol, and PEG-7 glyceryl cocoate.

The product may also contain one or more further ingredients selected from the following:

Stabilisers for stabilising the foam of the surfactant, for example, coconut diethanolamide, which enables a satisfactory foam to be obtained notwithstanding the presence of sebum;

A substance which is bitter to the taste in order to discourage ingestion of the product by the user e.g. children. (This could be denatonium benzoate which is known by its trade name of Bitrex.);

Oils, which are included for their lubricating properties, giving improved sensory qualities, and as moisturisers;

Colourings, especially cosmetic grade synthetic or vegetable colourings;

Pigments, for example, mica pigments;

Pearling agents;

Fragrances;

UV stabilisers such as benzophenone-1; and

Emulsifiers, for example, cetyl stearyl alcohol.

The foam booster/stabiliser is preferably Cocamide DEA. It allows a foam to be produced even when a considerable quantity of dirt and oil are present in the water.

The products according to the invention may be made by dissolving gelatin in water, adding the surfactant and any other ingredients to the gelatin/water mixture at a temperature higher than ambient temperature, forming the mixture into the desired shape, and cooling the mixture to a setting temperature at which the mixture sets to form a gel.

The present invention provides a method of making a shaped toiletry product, comprising combining a gelling agent with an amount of water sufficient to form a gel, heating the mixture obtained to a temperature not exceeding ninety-eight degrees centigrade, and preferably not exceeding eighty-five degrees centigrade, adding to the mixture before or after said heating step at least one surfactant, placing the mixture in a mould, and cooling the mixture to form a shaped product. In an especially preferred embodiment the gelatin is dissolved in water that is heated to a temperature of between 55 and 65 degrees centigrade, and, after dissolving the gelatin, the surfactants are added. Adding the surfactants after dissolving the gelatin, provides the advantages of (i) saving energy when heating the water and gelatin, and (ii) assisting cooling of the mixture.

Advantageously, after dissolving the gelatin, the mixture is cooled to a temperature of between forty and fifty degrees centigrade before adding a perfume, and a colour. Preferably, the mixture is cooled to a temperature of between forty and forty five degrees centigrade before the perfume is added.

The shaped product may then be removed from the mould and, preferably, placed or wrapped in suitable packaging. If desired, however, the mixture may be placed in a mould that is suitable for inclusion in the packaging in which the shaped product is to be sold. In that case, after the mixture is set, the mould containing the shaped product is preferably packed in a suitable container. It has been found that the use of such a mould, which encloses the product, is advantageous in that it reduces the occurrence of moisture loss from the product.

In accordance with the invention, the gel may be allowed to set in any desired shape, for example, resembling the shape of an animal or another, inanimate object that might be regarded as appealing by consumers, including children.

### EXAMPLE 1

Cold water (19.2%) was placed in a mixing receptacle and, gelatin powder (5%; lime processed; Bloom strength 250 g) was added. One hour was then allowed for the gelatin to wet out. The sodium laureth sulphate of the following ingredients was then added and stirring of the mixture was started. The other ingredients listed below were then added to the mixture.

| | |
|---|---|
| Sodium Laureth Sulphate (add first) | (60%) |
| Coconut Diethonolamide | (5%) |
| Glycerine | (10%) |
| Bitrex (Denatonium Benzoate) | (0.2%) |
| Phenonip (Phenoxyethonol, methylparaben, | (0.3%) |
| ethylparaben, butylparaben, propylparaben, | |
| isobutlparaben) | |

The mixture was heated to about sixty-five degrees centigrade, whilst stirring gently. Strring was continued until the mixture became homogeneous and: clear apart from air bubbles. Colouring as required and a fragrance (0.3%) were added and the product was allowed to stand until the air bubbles had escaped. The mixture was then placed in moulds and allowed to cool. After cooling, it was found that the. mixture had set to form a shaped article, which could be removed from the mould.

The shaped bath gel obtained dissipated rapidly in water at forty degrees centigrade and gave excellent lather.

The dissolution characteristics of the bath gel of Example 1 were found to be especially good.

The product of Example 1 is designed to be enclosed in substantially air tight packaging to ensure that prior to its actual use it does not dehydrate to the point where the product is unusable.

### EXAMPLE 2

This example differs significantly from that of Example 1 in that it is intended that the final product will be capable of retaining its original characteristics without the need of being enclosed or wrapped in packaging to prevent its dehydration.

In this second example no separately introduced water is utilised, the gelatin being instead dissolved in the sodium laureth sulphate.

The ingredients are as set out below.

| | | |
|---|---|---|
| 1 | Gelatin (250 Bloom lime processed) | (5%) |
| 2 | Sodium laureth sulphate | (60%) |
| 3 | Glycerine | (29.2%) |
| 4 | Cocamide DEA | (5%) |
| 5 | Denatonium benzoate (Bitrex) | (0.2%) |
| 6 | Phenonip (Phenoxyethanol, methylparaben, | (0.3) |
| | ethylparaben, butylparaben, propylparaben, | |
| | isobutlparaben) | |
| 7 | Colour | (As required) |
| 8 | Fragrance | (0.3%) |

As indicated earlier the sodium laureth sulphate is first placed in the mixing vessel and the gelatin granules added to it so that the gelatin wets out or starts dissolving in the sodium laureth sulphate. One hour is allowed for this initial step.

The glycerine is then added to the sodium laureth sulphate/gelatin mixture followed by the ingredients listed at 4 to 6 above.

The mixture thus formed is then heated to approximately sixty-five degrees centigrade whilst being slowly stirred. All the ingredients dissolve and the product becomes homogenous and clear apart from air bubbles. The colour and fragrance (items 7 and 8 above) are added. The heat is then lowered and the product is allowed to stand for a short time until substantially all the air has escaped from it. The product can then be poured into moulds and cooled to allow the solid gel to form.

It was found that the shape was well preserved, without any significant change in texture due to evaporation of moisture, even after storing for a number of weeks at ambient temperature in air.

### EXAMPLE 3

This example differs from the first two examples in that it includes amphoteric surfactants and proportionately less anionic surfactant, to provide a milder effect on the skin. In addition, the quantity of gelatin is relatively less, providing the product with faster and easier dissolving in bath water, as well as reducing the time required to dissolve the gelatin when making the product. This example also includes an alternative humectant, PEG-7 glyceryl cocoate, and a colour stabiliser benzophenone-1.

The ingredients are as set out below.

The ingredients are as set out below.

| | Ingredient | % w/w |
|---|---|---|
| A1 | De-ionised water | ˜26(to 100%) |
| A2 | Denatonium Benzoate, 2.5% solution | 0.04 |
| A3 | Phenochem (phenoxyethanol, | 0.6 |
| | methyl-paraben, butyl-paraben, | |
| | ethyl-paraben, propyl-paraben) | |
| A4 | Gelatin | 4.2 |
| B5 | Cocamidopropyl Betaine, 30% solution | 6 |
| B6 | Sodium Cocoamphoacetate, 32% solution | 4 |
| B7 | Sodium Laureth Sulphate, 28% solution | 50 |
| C8 | Cocamide DEA | 4.2 |
| C9 | Fragrance | 1 |
| C10 | PEG-7 Glyceryl Cocoate | 3 |
| C11 | Benzophenone-1 | 0.2 |
| D12 | Citric Acid, 25% solution | as required |
| E13 | Colours | as required |
| | (FD & C Blue 1, 1% solution) | (˜0.015%) |
| | (D & C Red 33, 1% solution) | (˜0.22%) |

To produce the product, the de-ionised water (A1), denatonium benzoate (A2), Phenochem (A3), and gelatin (A4) are mixed together with constant stirring, allowing the gelatin to be wefted out. When the gelatin is thoroughly wefted out, the mixture is heated to 60 degrees centigrade to dissolve the gelatin. This may, for example, be carried out in a suitably sized stainless steel jacketed pan.

When the gelatin is fully dissolved, the heat is turned off, and each of the ingredients B5, B6 and B7 (cocamidopropyl betaine, sodium cocoamphoacetate, and sodium laureth sulphate) are added separately to the mixture. The mixture is then cooled to 40 degrees centigrade before further components are added.

The ingredients C8, C9, C10 and C11 are mixed together and stirred until the fragrance (C9) and benzophenone-1 (C11) are fully dissolved in the PEG-7 glyceryl cocoate (C10) and cocamide DEA (C8). This mixture is then added to the main mixture which is maintained at 40 degrees centigrade.

Citric acid (D12) is added to adjust the pH value as required. A pH of 6 is typically produced.

Lastly, one or more colours (E13) are added to provide the desired appearance of the final product.

Finally the mixture which is still at 40 degrees centigrade is poured into moulds and allowed to cool and set. When set the shaped toiletry product is removed from the mould.

1-21. Cancelled

21. A toiletry product in tablet form comprising:
a surfactant;
gelatin; and
a non-formaldehyde preservative,
characterized in that the product is substantially free from formaldehyde.

22. A toiletry product as claimed in claim 21 including one or more of:
water;
a foam booster/stabilizer;
a moisturizer/humectant;
an anti-ingester;
a coloring;
a fragrance.

23. A toiletry product as claimed in claim 21 in which the gelatin has a Bloom strength of from about 150 to 270 g.

24. A toiletry product according to claim 21 in which the surfactant is an anionic surfactant.

25. A toiletry product according to claim 24 in which the surfactant is selected from sodium lauryl sulfate, triethanolamine lauryl sulfate, sodium lauryl ethersulfate, ammonium lauryl ethersulfate and monoethanolamine lauryl sulfate.

26. A toiletry product as claimed in claim 21 in which said surfactant comprises an amphoteric surfactant.

27. A toiletry product as claimed in claim 21 which said surfactant comprises a cationic surfactant.

28. A toiletry product as claimed in claim 22 in which the humectant is selected from glycerine and PEG-7 glyceryl cocoate.

29. A toiletry product as claimed in claim 22 further comprising an ultraviolet stabilizer.

30. A toiletry product as claimed in claim 21 in which the preservative contains parabens and phenoxyethanol.

31. A toiletry product as claimed in claim 21, wherein said gelatin comprises between about 4% and 4.5% of the mass of the product.

32. A method of making a shaped toiletry product containing one or more surfactants, which comprises the steps of:
dissolving gelatin in either water or a surfactant to form a mixture;
heating and stirring the mixture to homogenize it;
pouring the mixture into a mold and allowing the poured mixture to solidify by cooling to form a shaped product, wherein a non-formaldehyde preservative is included within the mixture and the constituents of the product are free from formaldehyde.

33. A method of making a shaped toiletry product which comprises the steps of:
mixing gelatin and water to form a mixture;
heating and stirring the water and gelatin mixture to fully dissolve the gelatin;
adding one or more surfactants to the mixture;
pouring the mixture into a mold and allowing the poured mixture to solidify by cooling in order to form a shaped product, wherein a non-formaldehyde preservative is included within the product, and each of the constituents of the product are free from formaldehyde.

34. A method according to claim 33, in which, after the gelatin has dissolved, the method comprises the steps of:
cooling the water and gelatin mixture to a temperature at which the mixture is still pourable;
adding one or more of:
(a) a foam booster;
(b) a perfume;
(c) a humectant; and
(d) a UV stabilizer.

35. A method according to claim 33, wherein said one or more surfactants include an anionic surfactant and an amphoteric surfactant.

36. A method according to claim 33, wherein said water and gelatin are heated to a temperature of between about 55 and 65 degrees centigrade.

37. A method according to claim 33, wherein the addition of said one or more surfactants assists cooling of the mixture.

38. A method according to claim 33, wherein said non-formaldehyde preservative is added to the water and gelatin before they are heated.

39. A method according to claim 33, wherein the non-formaldehyde preservative contains parabens and phenoxyethanol.

40. A method according to claim 33, wherein said gelatin comprises between about 4% and 4.5% of the mass of the product.
